(19) United States
(12) Patent Application Publication
(10) Pub. No.: US 2007/0149557 A1
(43) Pub. Date: Jun. 28, 2007

(54) CXCR3 antagonists

(75) Inventors: Tassie L. Collins, San Mateo, CA (US); Julio C. Medina, San Carlos, CA (US); Feng Xu, Palo Alto, CA (US); Liusheng Zhu, Burlingame, CA (US)

Correspondence Address: AMGEN INC., 1120 VETERANS BOULEVARD, SOUTH SAN FRANCISCO, CA, 94080, US

(73) Assignee: Amgen Inc., Thousand Oaks, CA (US)

(21) Appl. No.: 11/603,699

(22) Filed: Nov. 21, 2006

Related U.S. Application Data
(60) Provisional application No. 60/738,685, filed on Nov. 21, 2005.

(51) Int. Cl. A61K 31/4747; C07D 471/14

(52) U.S. Cl. 514/278; 546/17

(57) ABSTRACT

Compounds, compositions and methods are provided that are useful in the treatment of conditions and diseases mediated by a chemokine receptor. In particular, the compounds of the invention modulate the CXCR3 chemokine receptor. The subject methods are useful for the treatment of inflammatory and immunoregulatory disorders and diseases, such as multiple sclerosis, rheumatoid arthritis and type I diabetes.

### CROSS-REFERENCE TO RELATED APPLICATIONS

This application claims the benefit of U.S. Provisional Application No. 60/738,685 filed Nov. 21, 2005, which is incorporated herein by reference in its entirety.

### BACKGROUND OF THE INVENTION

Chemokines are chemotactic cytokines that are released by a wide variety of cells to attract macrophages, T cells, eosinophils, basophils and neutrophils to sites of inflammation (reviewed in Schall, Cytokine, 3:165-183 (1991), Schall, et al., Curr. Opin. Immunol., 6:865-873 (1994) and Murphy, Rev. Immun., 12:593-633 (1994)). In addition to stimulating chemotaxis, other changes can be selectively induced by chemokines in responsive cells, including changes in cell shape, transient rises in the concentration of intracellular free calcium ions ([Ca²⁺])ᵢ, granule exocytosis, integrin upregulation, formation of bioactive lipids (e.g., leukotrienes) and respiratory burst, associated with leukocyte activation. Thus, the chemokines are early triggers of the inflammatory response, causing inflammatory mediator release, chemotaxis and extravasation to sites of infection or inflammation.

There are four classes of chemokines, CXC (α), CC (β), C(γ), and CX₃C (δ), depending on whether the first two cysteines are separated by a single amino acid (C—X—C), are adjacent (C—C), have a missing cysteine pair (C), or are separated by three amino acids (CXC₃). The α-chemokines, such as interleukin-8 (IL-8), melanoma growth stimulatory activity protein (MGSA), and stromal cell derived factor 1 (SDF-1) are chemotactic primarily for neutrophils and lymphocytes, whereas β-chemokines, such as RANTES, MIP-1α, MIP-1β, monocyte chemotactic protein-1 (MCP-1), MCP-2, MCP-3 and eotaxin are chemotactic for macrophages, T-cells, eosinophils and basophils (Deng, et al., Nature, 381:661-666 (1996)). The C chemokine lymphotactin shows specificity for lymphocytes (Kelner, et al., Science, 266:1395-1399 (1994)) while the CX₃C chemokine fractalkine shows specificity for lymphocytes and monocytes (Bazan, et al., Nature, 385:640-644 (1997).

Chemokines bind specific cell-surface receptors belonging to the family of G-protein-coupled seven-transmembrane-domain proteins (reviewed in Horuk, Trends Pharm. Sci., 15:159-165 (1994)) which are termed “chemokine receptors.” On binding their cognate ligands, chemokine receptors transduce an intracellular signal through the associated heterotrimeric G protein, resulting in a rapid increase in intracellular calcium concentration. There are at least twelve human chemokine receptors that bind or respond to β-chemokines with the following characteristic pattern: CCR1 (or “CKR-1” or “CC-CKR-1”) MIP-1α, MIP-1β, MCP-3, RANTES (Ben-Barruch, et al., J. Biol. Chem., 270:22123-22128 (1995); Neote, et al., Cell, 72:415-425 (1993)); CCR2A and CCR2B (or “CKR-2A”/“CKR-2A” or “CC-CKR-2A”/“CC-CKR2A”) MCP-1, MCP-3, MCP-4; CCR3 (or “CKR-3” or “CC-CKR-3”) eotaxin, RANTES, MCP; (Ponath, et al., J. Exp. Med., 183:2437-2448 (1996)); CCR4 (or “CKR-4” or “CC-CKR-4”) TARC, MDC (Imai, et al., J. Biol. Chem., 273:1764-1768 (1998)); CCR5 (or “CKR-5” or “CC-CKR-5”) MIP-1α, RANTES, MIP-1β (Sanson, et al., Biochemistry, 35:3362-3367 (1996)); CCR6 MIP-3 alpha (Greaves, et al., J. Exp. Med., 186:837-844 (1997)); CCR7 MIP-3 beta and 6Ckine (Campbell, et al., J. Cell. Biol., 141:1053-1059(1998)); CCR8 I-309, HHV8 vMIP-I, HHV-8 vMIP-II, MCV vMCC-I (Dairaghi, et al., J. Biol. Chem., 274:21569-21574 (1999)); CCR9 TECK (Zaballos, et al., J. Immunol., 162:5671-5675 (1999)), D6 MIP-1 beta, RANTES, and MCP-3 (Nibbs, et al., J. Biol. Chem., 272:32078-32083 (1997)), and the Duffy blood-group antigen RANTES, MCP-1 (Chaudhun, et al., J. Biol. Chem., 269:7835-7838 (1994)).

Chemokine receptors, such as CCR1, CCR2, CCR2A, CCR2B, CCR3, CCR4, CCR5, CCR6, CCR7, CCR8, CCR9, CXCR1, CXCR2, CXCR3, CXCR4, CXCR5, CX₃CR1, and XCR1 have been implicated as being important mediators of inflammatory and immunoregulatory disorders and diseases, including asthma and allergic diseases, as well as autoimmune pathologies such as rheumatoid arthritis and atherosclerosis.

The CXCR3 chemokine receptor is expressed primarily in T lymphocytes, and its functional activity can be measured by cytosolic calcium elevation or chemotaxis. The receptor was previously referred to as GPR9 or CKR-L2. Its chromosomal location is unusual among the chemokine receptors in being localized to Xq13. Ligands that have been identified that are selective and of high affinity are the CXC chemokines, IP10, MIG and ITAC.

The highly selective expression of CXCR3 makes it an ideal target for intervention to interrupt inappropriate T cell trafficking. The clinical indications for such intervention are in T-cell mediated autoimmune diseases such as multiple sclerosis, rheumatoid arthritis, and type I diabetes. Inappropriate T-cell infiltration also occurs in psoriasis and other pathogenic skin inflammation conditions, although the diseases may not be true autoimmune disorders. In this regard, up-regulation of IP-10 expression in keratinocytes is a common feature in cutaneous immunopathologies. Inhibition of CXCR3 can be beneficial in reducing rejection in organ transplantation. Ectopic expression of CXCR3 in certain tumors, especially subsets of B cell malignancies indicate that selective inhibitors of CXCR3 will have value in tumor immunotherapy, particularly attenuation of metastasis.

In view of the clinical importance of CXCR3, the identification of compounds that modulate CXCR3 function represents an attractive avenue into the development of new therapeutic agents. Such compounds are provided herein.

### SUMMARY OF THE INVENTION

The present invention is directed to compounds which are modulators of CXCR3 chemokine receptor activity and are useful in the prevention or treatment of certain inflammatory and immunoregulatory disorders and diseases, including asthma and allergic diseases, as well as autoimmune pathologies such as rheumatoid arthritis and atherosclerosis. The invention is also directed to pharmaceutical compositions comprising these compounds and the use of these compounds and compositions in the prevention or treatment of diseases in which CXCR3 chemokine receptors are involved.

More particularly, the compounds provided herein have the general formula: 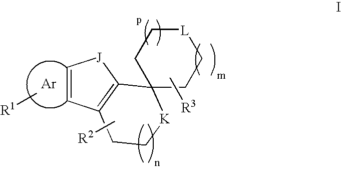
including enantiomers, diastereomers, salts and solvates thereof
wherein
Ar is an aryl or heteroaryl ring system;
R¹, R² and R³ are each one or more optional substituents as allowed by valance at each occurrence being independently selected from
   (i) halo, nitro, cyano, keto, alkyl, aryl, heteroaryl, heteroalkyl, cylcoalkyl, —NR′R′, —OR*, —C(═O)R⁺, —C(═O)OR*, —C(═O)NR′R′, —N(R′)—C(═O)(R⁺), —NR′S(O)ₓAr, —S(O)ₓAr, or —S(O)ₓNR′R′
   (ii) alkyl, alkenyl, alkynyl, aryl, heteroaryl, or heteroalkyl optionally substituted with one or more groups (i) above as allowed by valence
R′ at each occurrence is independently H, alkyl, aryl, heteroaryl, hetoralkyl, cycloalkyl, (aryl)alkyl, (heteroaryl)alkyl, (cycloalkyl)alkyl, (heteroalkyl)alkyl;
R* at each a occurrence is H, alkyl, aryl, heteroaryl, hetoralkyl, cycloalkyl, (aryl)alkyl, (heteroaryl)alkyl, (cycloalkyl)alkyl, (heteroalkyl)alkyl;
R⁺ at each occurrence is H, alkyl, aryl, heteroaryl, hetoralkyl, cycloalkyl, (aryl)alkyl, (heteroaryl)alkyl, (cycloalkyl)alkyl, (heteroalkyl)alkyl;
J is NR⁴, CR⁵R⁶, O or S;
K is NR^{4a}, CR^{5a}R^{6a};
L is NR⁷ or CR^{5b}R^{6b};
R⁴ and R^{4a} are independently H, alkyl, aryl, heteroaryl, heteroalkyl, cycloalkyl, (aryl)alkyl, (heteroaryl)alkyl, (cycloalkyl)alkyl, (heteroalkyl)alkyl
wherein groups other than hydrogen are optionally independently substituted with one or more alkyl, aryl, heteroaryl, hetoralkyl, cycloalkyl, cycloheteroalkyl, (aryl)alkyl, (heteroaryl)alkyl, (cycloalkyl)alkyl, (cycloheteroalkyl)alkyl;
R⁵, R⁶, R^{5a}, R^{6a}, R^{5b} and R^{6b} are independently
   (a) H, halo, —NR′R′, —OR*, —C(═O)R⁺, —C(═O)OR*, —C(═O)NR′R′, or —N(R′)—C(═O)(R⁺); or
   (b) alkyl, alkenyl, alkynyl, aryl, heteroaryl, or heteroalkyl optionally substituted with one or more groups (a) above as allowed by valence
R⁷ is
   (i) H,
   (ii) alkyl, cycloalkyl, heteroalkyl, cycloheteroalkyl, aryl, heteroaryl, any of which may be optionally substituted with one or more
      halo, nitro, cyano, keto, aryl, heteroaryl, cylcoalkyl, cycloheteroalkyl, —NR′R′, —OR*, —C(═O)R⁺, C(═O)OR*, C(═O)NR′R′, or —N(R′)—C(═O)(R⁺)
n is 0, 1, 2 or 3
m and p are each independently 0, 1, 2 or 3 provided that m and p can not each be 0
x at each occurrence is independently 0, 1 or 2

### DETAILED DESCRIPTION OF THE INVENTION

Definitions

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkylene” by itself or as part of another substituent means a divalent radical derived from an alkane, as exemplified by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms, or more.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively. Similarly, the term dialkylamino refers to an amino group having two attached alkyl groups that can be the same or different.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. When a prefix such as (C₂-C₈) is used to refer to a heteroalkyl group, the number of carbons (2-8, in this example) is meant to include the heteroatoms as well. For example, a C₂-heteroalkyl group is meant to include, for example, —CH₂OH (one carbon atom and one heteroatom replacing a carbon atom) and —CH₂SH. The term “heteroalkylene” by itself or as part of another substituent means a divalent radical derived from heteroalkyl, as exemplified by —CH₂—CH₂—S—CH₂CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include trifluoromethyl, 2,2,2-trifluoroethyl, 4-chlorobutyl, 3-bromopropyl, and the like.

The term “aryl” means, unless otherwise stated, a polyunsaturated, typically aromatic, hydrocarbon substituent which can be a single ring or multiple rings (up to three rings) which are fused together or linked covalently. The term “heteroaryl” refers to aryl groups (or rings) that contain from zero to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″, —SR′, —halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —CN and —NO₂ in a number ranging from zero to (2m′+1), where m′ is the total number of carbon atoms in such radical. R′, R″ and R′″ each independently refer to hydrogen, unsubstituted (C₁-C₈)alkyl and heteroalkyl, unsubstituted aryl, aryl substituted with 1-3 halogens, alkoxy or thioalkoxy groups, or aryl-(C₁-C₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. From the above discussion of substituents, one of skill in the art will understand that the term “alkyl” in its broadest sense is meant to include groups such as haloalkyl (e.g., —CF₃ and —CH₂CF₃) and acyl (e.g., —C(O)CH₃, —C(O)CF₃, —C(O)CH₂OCH₃, and the like). Preferably, the alkyl groups will have from 0-3 substituents, more preferably 0, 1, or 2 substituents, unless otherwise specified.

Similarly, substituents for the aryl and heteroaryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted (C₁-C₆)alkyl.

As used herein, the term “heteroatom” is meant to include oxygen (O), nitrogen (N), sulfur (S) and silicon (Si).

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, et al. (1977) J. Pharm. Sci. 66:1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. Prodrugs are often useful because, in some situations, they may be easier to administer than the parent drug. They may, for instance, be bioavailable by oral administration whereas the parent drug is not. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present invention which is administered as an ester (the “prodrug”), but then is metabolically hydrolyzed to the carboxylic acid, the active entity. Additional examples include peptidyl derivatives of a compound of the invention.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

### DESCRIPTION OF THE EMBODIMENTS

The present invention is directed to compounds, compositions and methods useful in the modulation of chemokine receptor activity, particularly CXCR3. Accordingly, the compounds of the present invention are those which inhibit at least one function or characteristic of a mammalian CXCR3 protein, for example, a human CXCR3 protein.

The ability of a compound to inhibit such a function can be demonstrated in a binding assay (e.g., ligand binding or agonist binding), a signaling assay (e.g., activation of a mammalian G protein, induction of rapid and transient increase in the concentration of cytosolic free calcium), and/or cellular response function (e.g., stimulation of chemotaxis, exocytosis or inflammatory mediator release by leukocytes).

CXCR3 Antagonists

The present invention provides new compounds that are useful as antagonists of CXCR3, having particular utility for the treatment of inflammation and other CXCR3-mediated disorders. The compounds provided herein have the general formula (I): 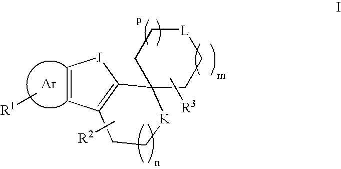
enantiomers, diastereomers, salst and solvates thereof
wherein
Ar is an aryl or heteroaryl ring system;
R¹, R² and R³ are each one or more optional substituents as allowed by valance at each occurrence being independently selected from
   (i) halo, nitro, cyano, keto, alkyl, aryl, heteroaryl, heteroalkyl, cylcoalkyl, —NR′R′, —OR*, —C(═O)R⁺, —C(═O)OR*, —C(═O)NR′R′, —N(R′)—C(═O)(R⁺), —NR′S(O)ₓAr, —S(O)ₓAr, or —S(O)ₓNR′R′
   (ii) alkyl, alkenyl, alkynyl, aryl, heteroaryl, or heteroalkyl optionally substituted with one or more groups (i) above as allowed by valence
R′ at each occurrence is independently H, alkyl, aryl, heteroaryl, hetoralkyl, cycloalkyl, (aryl)alkyl, (heteroaryl)alkyl, (cycloalkyl)alkyl, (heteroalkyl)alkyl;
R* at each a occurrence is H, alkyl, aryl, heteroaryl, hetoralkyl, cycloalkyl, (aryl)alkyl, (heteroaryl)alkyl, (cycloalkyl)alkyl, (heteroalkyl)alkyl;
R⁺ at each occurrence is H, alkyl, aryl, heteroaryl, hetoralkyl, cycloalkyl, (aryl)alkyl, (heteroaryl)alkyl, (cycloalkyl)alkyl, (heteroalkyl)alkyl;
J is NR⁴, CR⁵R⁶, O or S;
K is NR^{4a}, CR^{5a}R^{6a};
L is NR⁷ or CR^{5b}R^{6b};
R⁴ and R^{4a} are independently H, —C(═O)R⁺, alkyl, aryl, heteroaryl, heteroalkyl, cycloalkyl, (aryl)alkyl, (heteroaryl)alkyl, (cycloalkyl)alkyl, (heteroalkyl)alkyl
wherein groups other than hydrogen are optionally independently substituted with one or more halo, hydroxyl, alkyl, alkoxy, cyano, nitro, aryl, heteroaryl, hetoralkyl, cycloalkyl, cycloheteroalkyl, (aryl)alkyl, (heteroaryl)alkyl, (cycloalkyl)alkyl, (cycloheteroalkyl)alkyl;
R⁵, R⁶, R^{5a}, R^{6a}, R^{5b} and R^{6b} are independently
   (a) H, halo, —NR′R′, —OR*, —C(═O)R⁺, —C(═O)OR*, —C(═O)NR′R′, or —N(R′)—C(═O)(R⁺); or
   (b) alkyl, alkenyl, alkynyl, aryl, heteroaryl, or heteroalkyl optionally substituted with one or more groups (a) above as allowed by valence
R⁷ is alkyl, cycloalkyl, heteroalkyl, cycloheteroalkyl, aryl, heteroaryl, any of which may be optionally substituted with one or more
   halo, nitro, cyano, keto, aryl, heteroaryl, alkyl, alkoxy, aryloxy, heteroaryloxy, heteroalkoxy, (hydroxy)alkyl, haloalkyl, (alkoxy)alkyl, cylcoalkyl, cycloheteroalkyl, —NR′R′, —OR*, C(═O)R⁺, C(═O)OR*, C(═O)NR′R′, or —N(R′)—C(═O)(R⁺)
n is 0, 1, 2 or 3
m and p are read independently 0, 1, 2 or 3 provided that m and p can not each be 0

Preferred compounds of the present invention include compounds of the following Formula II

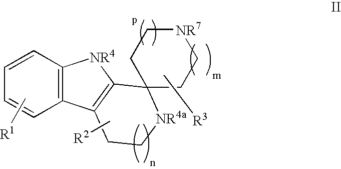

wherein
R⁴ and R^{4a} are independently H, alkyl, alkenyl, alkynyl, or —C(═O)R⁺; and
R⁷ is alkyl, (aryl)alkyl, (heteroaryl)alkyl, (heterocyclo)alkyl, aryl, heteroaryl, or heterocylo wherein groups other than hydrogron are optionally substituted with one or more
   halo, nitro, cyano, haloalkyl, cycloheteroalkyl, —NR′R′, —OR*, —C(═O)R⁺, C(═O)OR*, C(═O)NR′R′, or —N(R′)—C(═O)(R⁺)

More preferred compounds of the present invention include compounds of the following Formula III 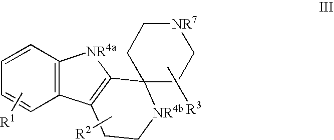
wherein
R⁴ and R^{4a} are independently H, alkyl, alkenyl, alkynyl, or —C(═O)R⁺; and R⁷ is phenyl or pyridyl (especially pyrid-2-yl) optionally independently substituted with one to three halo, cyano, nitro, —NR′R′, —OR*, —C(═O)R⁺, C(═O)OR*, C(═O)NR′R′, or —N(R′)—C(═O)(R⁺)
   Compositions of CXCR3 Antagonists

In another aspect, the present invention provides compositions for modulating chemokine receptor activity in humans and animals. The compositions comprise a compound of the present invention with a pharmaceutically acceptable carrier or diluent.

“Modulation” or modulating of chemokine receptor activity, as used herein in its various forms, is intended to encompass antagonism, agonism, partial antagonism and/or partial agonism of the activity associated with a particular chemokine receptor, preferably the CXCR3 receptor. The term “composition” as used herein is intended to encompass a product comprising the specified ingredients (and in the specified amounts, if indicated), as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The pharmaceutical compositions for the administration of the compounds of this invention may conveniently be presented in unit dosage form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108; 4,166,452 and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. As used herein, topical application is also meant to include the use of mouth washes and gargles.

The pharmaceutical composition and method of the present invention may further comprise other therapeutically active compounds as noted herein which are usually applied in the treatment of the above mentioned pathological conditions.

Methods of Treating or Preventing CXCR3-Mediated Conditions or Diseases

In yet another aspect, the present invention provides methods of treating or preventing CXCR3-mediated conditions or diseases by administering to a subject having such a disease or condition, a therapeutically effective amount of a compound of formula I above. The “subject” is defined herein to include animals such as mammals, including, but not limited to, primates (e.g., humans), cows, sheep, goats, horses, dogs, cats, rabbits, rats, mice and the like.

As used herein, the phrase “CXCR3-mediated condition or disease” and related phrases and terms refer to a condition characterized by inappropriate, e.g., less than or greater than normal, CXCR3 activity. Inappropriate CXCR3 activity might arise as the result of CXCR3 expression in cells which normally do not express CXCR3, increased CXCR3 expression (leading to, e.g., inflammatory and immunoregulatory disorders and diseases), or, decreased CXCR3 expression (leading to, e.g., certain cancers and angiogenic and vasculogenic-related disorders). Inappropriate CXCR3 functional activity might arise as the result of CXCR3 expression in cells which normally do not express CXCR3, increased CXCR3 expression (leading to, e.g., inflammatory and immunoregulatory disorders and diseases) or decreased CXCR3 expression. Inappropriate CXCR3 functional activity might also arise as the result of chemokine secretion by cells which normally do not secrete a CXC chemokine, increased chemokine expression (leading to, e.g., inflammatory and immunoregulatory disorders and diseases) or decreased chemokine expression. A CXCR3-mediated condition or disease may be completely or partially mediated by inappropriate CXCR3 functional activity. However, a CXCR3-mediated condition or disease is one in which modulation of CXCR3 results in some effect on the underlying condition or disease (e.g., a CXCR3 antagonist results in some improvement in patient well-being in at least some patients).

The term “therapeutically effective amount” means the amount of the subject compound that will elicit the biological or medical response of a tissue, system, animal or human that is being sought by the researcher, veterinarian, medical doctor or other clinician or that is sufficient to prevent development of or alleviate to some extent one or more of the symptoms of the disease being treated.

Diseases and conditions associated with inflammation, infection and cancer can be treated with the present compounds and compositions. In one group of embodiments, diseases or conditions, including chronic diseases, of humans or other species can be treated with inhibitors of CXCR3 function. These diseases or conditions include: (1) inflammatory or allergic diseases such as systemic anaphylaxis or hypersensitivity responses, drug allergies, insect sting allergies and food allergies; inflammatory bowel diseases, such as Crohn's disease, ulcerative colitis, ileitis and enteritis; vaginitis; psoriasis and inflammatory dermatoses such as dermatitis, eczema, atopic dermatitis, allergic contact dermatitis, urticaria; vasculitis; spondyloarthropathies; scleroderma; asthma and respiratory allergic diseases such as allergic rhinitis, hypersensitivity lung diseases, and the like, (2) autoimmune diseases, such as arthritis (rheumatoid and psoriatic), multiple sclerosis, systemic lupus erythematosus, type I diabetes, glomerulonephritis, and the like, (3) graft rejection (including allograft rejection and graft-v-host disease) and conditions associated therewith, and (4) other diseases in which undesired inflammatory responses are to be inhibited, e.g., atherosclerosis, myositis, neurodegenerative diseases (e.g., Alzheimer's disease), encephalitis, meningitis, hepatitis, nephritis, sepsis, sarcoidosis, conjunctivitis, otitis, chronic obstructive pulmonary disease, sinusitis and Behcet's syndrome. In another group of embodiments, diseases or conditions are treated or prevented with agonists of CXCR3 function. Examples of diseases to be treated or prevented with CXCR3 agonists include cancers, diseases in which angiogenesis or neovascularization play a role (neoplastic diseases, retinopathy and macular degeneration), infectious diseases and immunosuppressive diseases.

Preferably, the present methods are directed to the treatment or prevention of diseases or conditions selected from neurodegenerative diseases (e.g., Alzheimer's disease), multiple sclerosis, systemic lupus erythematosus, rheumatoid arthritis, atherosclerosis, encephalitis, meningitis, hepatitis, nephritis, sepsis, sarcoidosis, psoriasis, eczema, uticaria, type I diabetes, asthma, conjunctivitis, otitis, allergic rhinitis, chronic obstructive pulmonary disease, sinusitis, dermatitis, inflammatory bowel disease, ulcerative colitis, Crohn's disease, Behcet's syndrome, gout, cancer, anti-proliferative disorders, viral infections (e.g., HIV), bacterial infections, and organ transplant conditions or skin transplant conditions. The term “organ transplant conditions” is meant to include bone marrow transplant conditions and solid organ (e.g., kidney, liver, lung, heart, pancreas or combination thereof) transplant conditions.

Diseases or conditions that can be treated or prevented with the present compounds and compositions include diseases commonly associated with (1) inflammatory or allergic diseases, (2) autoimmune diseases, (3) graft rejection and (4) other diseases in which undesired inflammatory responses are to be inhibited, as described above. For example, restenosis following a procedure such as balloon angioplasty, is commonly associated with atherosclerosis and can be treated with the present compounds and compositions.

Depending on the disease to be treated or prevented and the subject's condition, the compounds of the present invention may be administered by oral, parenteral (e.g., intramuscular, intraperitoneal, intravenous, ICV, intracisternal injection or infusion, subcutaneous injection, or implant), inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

In the treatment or prevention of conditions which require chemokine receptor modulation an appropriate dosage level will generally be about 0.001 to 100 mg per kg patient body weight per day which can be administered in single or multiple doses. Preferably, the dosage level will be about 0.01 to about 25 mg/kg per day; more preferably about 0.05 to about 10 mg/kg per day. A suitable dosage level may be about 0.01 to 25 mg/kg per day, about 0.05 to 10 mg/kg per day, or about 0.1 to 5 mg/kg per day. Within this range the dosage may be 0.005 to 0.05, 0.05 to 0.5 or 0.5 to 5.0 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The compounds of the present invention can be combined with other compounds having related utilities to prevent and treat inflammatory and immunoregulatory disorders and diseases, including asthma and allergic diseases, as well as autoimmune pathologies such as rheumatoid arthritis and atherosclerosis, and those pathologies noted above.

For example, in the treatment or prevention of inflammation, the present compounds may be used in conjunction with an anti-inflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal anti-inflammatory agent, or a cytokine-suppressing anti-inflammatory agent, for example with a compound such as acetaminophen, aspirin, codeine, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextromethorphan; a diuretic; and a sedating or non-sedating antihistamine. Likewise, compounds of the present invention may be used in combination with other drugs that are used in the treatment/prevention/suppression or amelioration of the diseases or conditions for which compounds of the present invention are useful. Such other drugs may be administered, by a route and in an amount commonly used therefore, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention. Examples of other active ingredients that may be combined with a compound of the present invention, either administered separately or in the same pharmaceutical compositions, include, but are not limited to: (a) VLA-4 antagonists, (b) steroids such as beclomethasone, methylprednisolone, betamethasone, prednisone, dexamethasone, and hydrocortisone; (c) immunosuppressants such as cyclosporine (cyclosporine A, Sandimmune®, Neoral®, tacrolimus (FK-506, Prograf®), rapamycin (sirolimus, Rapamune®) and other FK-506 type immunosuppressants, and mycophenolate, e.g., mycophenolate mofetil (CellCept®); (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (e) non-steroidal anti-asthmatics such as .beta.2-agonists (terbutaline, metaproterenol, fenoterol, isoetharine, albuterol, bitolterol, and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal anti-inflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors such as celecoxib (Celebrex®) and rofecoxib (Vioxx®); (h) inhibitors of phosphodiesterase type IV (PDE-IV); (i) gold compounds such as auranofin and aurothioglucose, 0) inhibitors of phosphodiesterase type IV (PDE-IV); (k) other antagonists of the chemokine receptors, especially CCR1, CCR2, CCR3, CCR5, CCR6, CCR8 and CCR10; (1) cholesterol lowering agents such as HMG-CoA reductase inhibitors (lovastatin, simvastatin and pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (m) anti-diabetic agents such as insulin, sulfonylureas, biguanides (metformin), α-glucosidase inhibitors (acarbose) and glitazones (troglitazone and pioglitazone); (n) preparations of interferon beta (interferon β-1 α, interferon β-1 β); (o) etanercept (Enbrel®), (p) antibody therapies such as orthoclone (OKT3), daclizumab (Zenapax®) and basiliximab (Simulect®); and (q) other compounds such as 5-aminosalicylic acid and prodrugs thereof, infliximab (Remicade®), hydroxychloroquine, D-penicillamine, antimetabolites such as azathioprene and 6-mercaptopurine, and cytotoxic cancer chemotherapeutic agents. The weight ratio of the compound of the present invention to the second active ingredient may be varied and will depend upon the effective dose of each ingredient. Generally, an effective dose of each will be used. Thus, for example, when a compound of the present invention is combined with an NSAID the weight ratio of the compound of the present invention to the NSAID will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

Immunosuppressants within the scope of the present invention further include, but are not limited to, leflunomide, RAD001, ERL080, FTY720, CTLA-4, antibody therapies such as orthoclone (OKT3), daclizumab (Zenapax®) and basiliximab (Simulect®), and antithymocyte globulins such as thymoglobulins.

In particularly preferred embodiments, the present methods are directed to the treatment of multiple sclerosis using a compound of the invention either alone or in combination with a second therapeutic agent selected from betaseron, avonex, azathioprene (Imurek®, Imuran®), capoxone, prednisolone and cyclophosphamide. When used in combination, the practitioner can administer a combination of the therapeutic agents, or administration can be sequential.

In still other particularly preferred embodiments, the present methods are directed to the treatment of rheumatoid arthritis, wherein the compound of the invention is administered either alone or in combination with a second therapeutic agent selected from the group consisting of methotrexate, sulfasalazine, hydroxychloroquine, cyclosporine A, D-penicillamine, infliximab (Remicade®), etanercept (Enbrel®), auranofin and aurothioglucose.

In yet other particularly preferred embodiments, the present methods are directed to the treatment of an organ transplant condition wherein the compound of the invention is used alone or in combination with a second therapeutic agent selected from the group consisting of cyclosporine A, FK-506, rapamycin, mycophenolate, prednisolone, azathioprene, cyclophosphamide and an antilymphocyte globulin.

Methods of Evaluating Putative CXCR3 Modulators

In yet another aspect, the present invention includes methods to evaluate putative specific agonists or antagonists of CXCR3 function. Accordingly, the present invention is directed to the use of these compounds in the preparation and execution of screening assays for compounds which modulate the activity of the CXCR3 chemokine receptor. For example, the compounds of this invention are useful for isolating receptor mutants, which are excellent screening tools for more potent compounds. Furthermore, the compounds of this invention are useful in establishing or determining the binding site of other compounds to the CXCR3 chemokine receptor, e.g., by competitive inhibition. The compounds of the instant invention are also useful for the evaluation of putative specific modulators of the CXCR3 chemokine receptor, relative to other chemokine receptors including CCR1, CCR2, CCR2A, CCR2B, CCR3, CCR4, CCR5, CCR6, CCR8, CCR10, CXCR3 and CXCR4. One of skill in the art will appreciate that thorough evaluation of specific agonists and antagonists of the above chemokine receptors has been hampered by the lack of availability of non-peptidyl (metabolically resistant) compounds with high binding affinity for these receptors. Thus the compounds provided herein are particularly useful in this context. Combinatorial libraries of putative CXCR3 agonists or antagonists can be screened for pharmacological activity in in vitro or in vivo assays. Conventionally, new chemical entities with useful properties are generated by identifying a chemical compound (called a “lead compound”) with some desirable property or activity, e.g., CXCR3 chemokine receptor modulation activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of potential therapeutic compounds (candidate compounds). Such “combinatorial chemical libraries” are then screened in one or more assays to identify those library members (particular chemical species or subclasses) that display a desired characteristic activity. The compounds thus identified can serve conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (i.e., the number of amino acids in a polypeptide compound). Millions of chemical compounds can be synthesized through such combinatorial mixing of chemical building blocks (Gallop et. al. (1994) J. Med. Chem. 37(9):1233-1251).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res. 37:487-493, Houghton et. al. (1991) Nature 354: 84-88), peptoid libraries (PCT Publication No WO 91/19735), encoded peptide libraries (PCT Publication WO 93/20242), random bio-oligomer libraries (PCT Publication WO 92/00091), benzodiazepine libraries (U.S. Pat. No. 5,288,514), libraries of diversomers, such as hydantoins, benzodiazepines and dipeptides (Hobbs et. al. (1993) Proc. Nat. Acad. Sci. USA 90:6909-6913), vinylogous polypeptide libraries (Hagihara et al. (1992) J. Amer. Chem. Soc. 114:6568), libraries of nonpeptidyl peptidomimetics with a Beta-D-Glucose scaffolding (Hirschmann et al. (1992) J. Amer. Chem. Soc. 114:9217-9218), analogous organic syntheses of small compound libraries (Chen et. al. (1994) J. Am. Chem. Soc. 116:2661), oligocarbamate libraries (Cho et al. (1993) Science 261:1303) and/or peptidyl phosphonate libraries (Campbell et al. (1994) J. Org. Chem. 59:658). See, generally, Gordon et al. (1994) J. Med. Chem. 37:1385-1401, nucleic acid libraries (see, e.g., Stratagene Corp.), peptide nucleic acid libraries (see, e.g., U.S. Pat. No. 5,539,083), antibody libraries (see, e.g., Vaughn et. al. (1996) Nature Biotechnology 14(3):309-314), and PCT/US96/10287), carbohydrate libraries (see, e.g., Liang et al. (1996) Science 274:1520-1522, and U.S. Pat. No. 5,593,853), and small organic molecule libraries (see, e.g., benzodiazepines, Baum (1993) C&EN January 18, page 33; isoprenoids, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514; and the like).

Devices for the preparation of combinatorial libraries are commercially available (see, e.g., 357 MPS, 390 MPS, Advanced Chem Tech, Louisville Ky.; Symphony, Rainin, Woburn Mass.; 433A Applied Biosystems, Foster City, Calif.; 9050 Plus, Millipore, Bedford, Mass.).

A number of well known robotic systems have also been developed for solution phase chemistries. These systems includes automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto Calif.), which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see e.g., ComGenex, Princeton N.J.; Asinex, Moscow, Russia; Tripos, Inc., St. Louis, Mo.; ChemStar, Ltd, Moscow, Russia; 3D Pharmaceuticals, Exton Pa.; Martek Biosciences, Columbia, Md.; etc.).

High throughput assays for the presence, absence, quantification, or other properties of particular compounds may be used to test a combinatorial library that contains a large number of potential therapeutic compounds (potential modulator compounds). The assays are typically designed to screen large chemical libraries by automating the assay steps and providing compounds from any convenient source to assays, which are typically run in parallel (e.g., in microtiter formats on microtiter plates in robotic assays). Preferred assays detect enhancement or inhibition of CXCR3 receptor function.

High throughput screening systems are commercially available (see e.g., Zymark Corp., Hopkinton Mass.; Air Technical Industries, Mentor Ohio; Beckman Instruments, Inc., Fullerton Calif.; Precision Systems, Inc., Natick Mass.; etc.). These systems typically automate entire procedures, including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, for example, Zymark Corp. provides technical bulletins describing screening systems for detecting the modulation of gene transcription, ligand binding, and the like.

### EXAMPLES

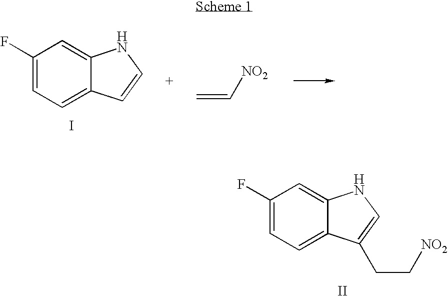

### 3-(2-Nitroethyl)-6-fluoroindole (II)

A mixture of 6-fluoroindole (6.6 mmol) and nitroethene (4.4 ml) in benzene (20 ml) were stirred at 0° C. to r.t. overnight. After concentration by rotavapor the residue was purified by column chromatography (silica gel, eluting with EtOAc/hexane). Product (II) was obtained in 40-50% yield.

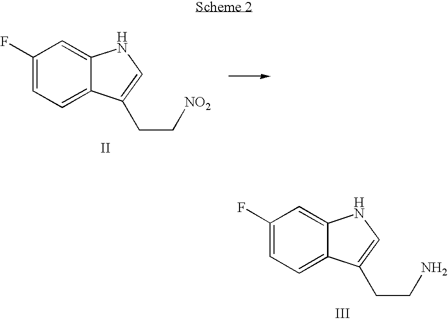

### 6-Fluorotryptamine (III)

A mixture of 3-(2-Nitroethyl)-7-methylindole (II) (2.8 mmol), hydrazine (8.4 mmol) and excessive Raney nickel in ethyl alcohol (10 ml) was stirred at r.t. for 1 hr. After filtration through celite and removal of the solvent, the residue was used for next step without purification

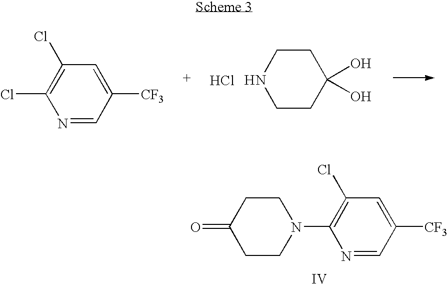

### 1-(3-Chloro-5-(trifluoromethyl)pyrid-2-yl)-4-piperidone (IV)

A solution of 2,3-dichloro-5-(trifluoromethyl)pyridine (32 mmol) and 4-piperidone hydrochloride monohydrate (32 mmol) in DMF (60 ml) was stirred at 70° C. overnight. After cooling, it was mixed with saturated NH₄Cl solution and extracted with ethyl ether. The organic phase was dried with MgSO₄ and the solvent was removed by rotavapor, obtaining crude product (IV) in 89% yield.

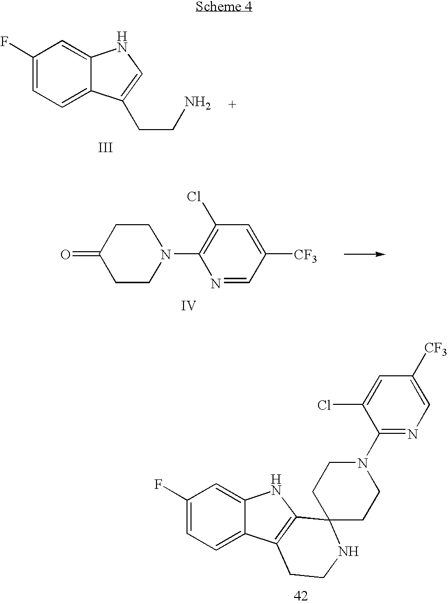

### Example (38)

To a solution of 6-fluorotryptamine (III) (1.2 mmol) and 1-(3-chloro-5-(trifluoromethyl)pyrid-2-yl)-4-piperidone (IV) (1.2 mmol) in chloroform (3 ml) was added excessive 4A molecular sieves. After refluxing for 1 hr, 4N hydrochloric acid (1.2 mmol) was added and it was refluxed overnight. Cooling to r.t., it was filtered through Celite. The filtration was neutralized with NaHCO₃ solution and extracted with chloroform. The organic phase was dried over anhydrous Na₂SO₄. After removing the solvent, the residue was purified by chromatography (silica gel, eluting with 1:9 methanol/dichloromethane with 2% NH₄OH), obtaining product (38) in 67% yield. MS ESI (positive) m/e: 439 (M+H). ¹H NMR (400 MHz) (CD₃OD) δ 11.00 (1H, s); 8.52 (1H, d, J=1.2 Hz); 8.05(1H, d, J=2.2 Hz); 7.46 (1H, m); 7.06 (1H, m); 6.86 (1H, m); 5.48 (1H, s); 4.15 (1H, d, J=14 Hz); 3.70 (2H, m); 3.46 (2H, m); 3.13 (2H, m); 2.65 (2H, m); 2.30 (2H, d, J=14 Hz).

TABLE 1

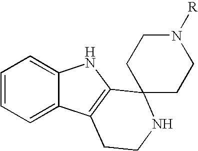

| | | ¹²⁵I-IP-10 |
|---|---|---|
| | | binding assay |
| Example | R | IC₅₀* |
| 1 | 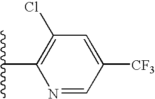 | + |
| 2 | 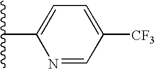 | +++ |
| 3 | 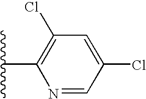 | +++ |
| 4 | 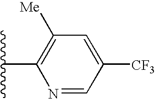 | +++ |
| 5 | 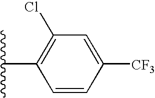 | + |
| 6 | 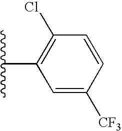 | +++ |
| 7 | 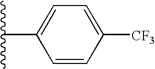 | +++ |
| 8 | 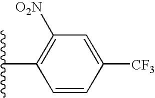 | +++ |
| 9 | 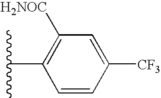 | ++ |
| 10 | 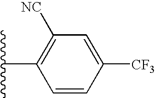 | +++ |
| 11 | 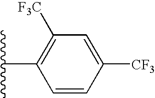 | +++ |
| 12 | 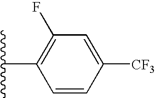 | +++ |
| 13 | 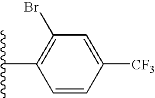 | +++ |
| 14 | 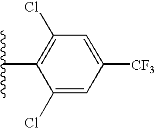 | +++ |
| 15 | 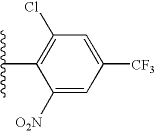 | + |
| 16 | 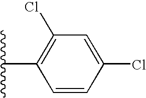 | +++ |
| 17 | 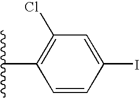 | +++ |
| 18 | 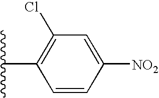 | +++ |
| 19 | 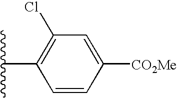 | +++ |

* + ≡ <500 nM
++ ≡ ≧500 nM, and ≦1 μM
+++ ≡ >1 μM

TABLE 2

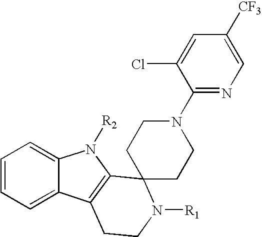

| Example | R₁ | R₂ |
|---|---|---|
| 20 | Me | H |
| 21 | Me | Me |
| 22 | 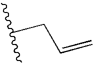 | H |
| 23 | 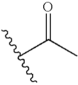 | H |

TABLE 3

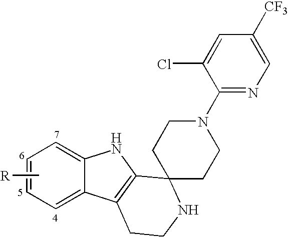

| Example | R |
|---|---|
| 24 | 4-OH |
| 25 | 4-Me |
| 26 | 4-OMe |
| 27 | 4-OCH₂Ph |
| 28 | 4-NH₂ |
| 29 | 4-F |
| 30 | 4-Cl |
| 31 | 4-CO₂Me |
| 32 | 5-Me |
| 33 | 5-OH |
| 34 | 5-OMe |
| 35 | 5-F |
| 36 | 5-Cl |
| 37 | 6-OMe |
| 38 | 6-F |
| 39 | 6-Cl |
| 40 | 6-Br |
| 41 | 7-Me |
| 42 | 7-Cl |

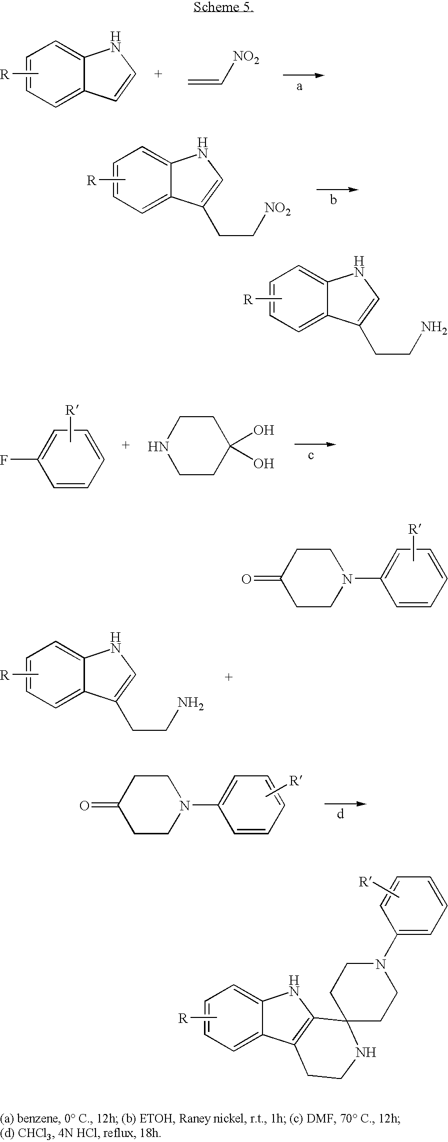

### Example (1)

MS ESI (positive) m/e: 421 (M+H). ¹H NMR (400 MHz) (CD₃OD) δ 8.42 (1H, m); 7.92 (1H, m); 7.36 (d, J=8 Hz); 7.26(1H, d, J=8 Hz); 7.0 (1H, m); 7.00 (1H, m); 6.92 (1H, m); 4.00 (2H, m); 3.40 (2H, m); 3.11 (2H, m); 2.71 (2H, m); 2.26-2.34 (2H, m); 1.86(2H, d, J=14 Hz).

### Example (5)

MS ESI (positive) m/e: 420 (M+H). ¹H NMR (400 MHz) (CD₃OD) δ 7.65 (1H, s); 7.56 (1H, m); 7.33-7.39 (2H, m); 7.28 (d, J=8 Hz); 7.02 (1H, m); 6.96 (1H, m); 3.34 (2H, m); (2H, m); 3.15-3.24 (4H, m); 2.75 (2H, m); 2.43 (2H, m); 1.95 (2H, d, J=13 Hz).

### Example (9)

MS ESI (positive) m/e: 429 (M+H). ¹H NMR (400 MHz) (CD₃OD) δ 8.06 (1H, m); 7.75 7.46 (d, J=9 Hz); 7.40 (d, J=8 Hz); 7.28 (d, J=8 Hz); 7.05 (1H, m); 5.96 (1H, m); 3.35 (2H, m); 3.23 (2H, m); 3.16 (2H, m); 2.74 (2H, m); 2.38 (2H, m); 1.97 (2H, d, J=13 Hz).

### Example (15)

MS ESI (positive) m/e: 465 (M+H). ¹H NMR (400 MHz) (CD₃OD) δ 8.09 (2H, m); 7.49 (1H, d, J=8 Hz); 7.37 (1H, d, J=8 Hz); 7.17 (1H, t, J=7 Hz); 7.07(1H, t, J=7 Hz); 3.69 (2H, m); 3.41(4H, m); 3.12 (2H, m); 2.70 (2H, m); 2.24 (2H, d, J=14 Hz).

### Example (25)

MS ESI (positive) m/e: 435 (M+H). ¹H NMR (400 MHz) (CD₃OD) δ 8.44 (1H, m); 7.95 (1H, m); 7.08 (1H, d, J=8 Hz); 8.87 (1H, m); 6.67 (1H, m); 4.06 (2H, m); 3.39 (2H, m); 3.19 (2H, m); 3.06 (2H, m); 2.59 (3H, s); 2.35 (2H, m); 1.93 (2H, m).

### Example (33)

MS ESI (positive) m/e: 437 (M+H). ¹H NMR (400 MHz) (CD₃OD) δ 8.42 (1H, m); 7.94 (1H, d, J=9 Hz); 6.77 (1H, m); 6.59 (1H, m); 4.00 (2H, d, J=13 Hz); 3.41 (2H, m); 3.30 (2H, m); 3.14 (2H, m); 2.67 (2H, m); 2.30 (2H, m); 1.90 (2H, d, J=13 Hz).

### Example (35)

MS ESI (positive) m/e: 439 (M+H). ¹H NMR (400 MHz) (CD₃OD) δ 8.43 (1H, m); 7.93 (1H, d, J=2 Hz); 7.20(1H, m); 7.04 (1H, m); 6.77 (1H, m); 4.04 (2H, m); 3.43 (2H, m); 3.16 (2H, m); 2.71 (2H, m); 2.32 (2H, m); 1.92 (2H, d, J=14 Hz).

### Example (41)

MS ESI (positive) m/e: 435 (M+H). ¹H NMR (400 MHz) (CD₃OD) δ 8.43 (1H, m); 7.93 (1H, m); 7.20 (d, J=8 Hz); 6.85 (2H, m); 4.07 (2H, m); 3.41 (2H, m); 3.30 (2H, m); 3.13 (2H, m); 2.71 (2H, m); 2.45 (3H, s); 1.88 (2H, d, J=14 Hz).

### Example (42)

¹H NMR (400 MHz) (CD₃OD) δ 8.43 (1H, m); 7.93 (1H, m); 7.33 (1H, m); 7.04 (d, J=8 Hz); 6.92 (1H, m); 4.06 (2H, m); 3.43 (2H, m); 3.14 (2H, m); 2.73 (2H, m); 2.47 (2H, m); 1.90 (2H, d, J=14 Hz).

What is claimed is:

1. A compound of Formula (I): 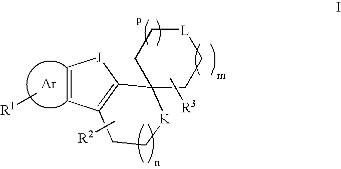
enantiomers, diastereomers, salst and solvates thereof
wherein
Ar is an aryl or heteroaryl ring system;
R¹, R² and R³ are each one or more optional substituents as allowed by valance at each occurrence being independently selected from
(i) halo, nitro, cyano, keto, alkyl, aryl, heteroaryl, heteroalkyl, cylcoalkyl, —NR′R′, —OR*, —C(═O)R⁺, —C(═O)OR*, —C(═O)NR′R′, —N(R′)—C(═O)(R⁺), —NR′S(O)ₓAr, —S(O)ₓAr, or —S(O)ₓNR′R′
(ii) alkyl, alkenyl, alkynyl, aryl, heteroaryl, or heteroalkyl optionally substituted with one or more groups (i) above as allowed by valence
R′ at each occurrence is independently H, alkyl, aryl, heteroaryl, hetoralkyl, cycloalkyl, (aryl)alkyl, (heteroaryl)alkyl, (cycloalkyl)alkyl, (heteroalkyl)alkyl;
R* at each a occurrence is H, alkyl, aryl, heteroaryl, hetoralkyl, cycloalkyl, (aryl)alkyl, (heteroaryl)alkyl, (cycloalkyl)alkyl, (heteroalkyl)alkyl;
R⁺ at each occurrence is H, alkyl, aryl, heteroaryl, hetoralkyl, cycloalkyl, (aryl)alkyl, (heteroaryl)alkyl, (cycloalkyl)alkyl, (heteroalkyl)alkyl;
J is NR⁴, CR⁵R⁶, O or S;
K is NR^{4a}, CR^{5a}R^{6a};
L is NR⁷ or CR^{5b}R^{6b};
R⁴ and R^{4a} are independently H, —C(═O)R⁺, alkyl, aryl, heteroaryl, heteroalkyl, cycloalkyl, (aryl)alkyl, (heteroaryl)alkyl, (cycloalkyl)alkyl, (heteroalkyl)alkyl
wherein groups other than hydrogen are optionally independently substituted with one or more halo, hydroxyl, alkyl, alkoxy, cyano, nitro, aryl, heteroaryl, hetoralkyl, cycloalkyl, cycloheteroalkyl, (aryl)alkyl, (heteroaryl)alkyl, (cycloalkyl)alkyl, (cycloheteroalkyl)alkyl;
R⁵, R⁶, R^{5a}, R^{6a}, R^{5b} and R^{6b} are independently
(a) H, halo, —NR′R′, —OR*, —C(═O)R⁺, —C(═O)OR*, —C(═O)NR′R′, or —N(R′)—C(═O)(R⁺); or
(b) alkyl, alkenyl, alkynyl, aryl, heteroaryl, or heteroalkyl optionally substituted with one or more groups (a) above as allowed by valence
R⁷ is alkyl, cycloalkyl, heteroalkyl, cycloheteroalkyl, aryl, heteroaryl, any of which may be optionally substituted with one or more
halo, nitro, cyano, keto, aryl, heteroaryl, alkyl, alkoxy, aryloxy, heteroaryloxy, heteroalkoxy, (hydroxy)alkyl, haloalkyl, (alkoxy)alkyl, cylcoalkyl, cycloheteroalkyl, —NR′R′, —OR*, —C(═O)R⁺, C(═O)OR*, C(═O)NR′R′, or —N(R′)—C(═O)(R⁺)
n is 0, 1, 2 or 3;
m and p are read independently 0, 1, 2 or 3 provided that m and p can not each be 0.

2. A compound of claim 1 wherein
R⁴ and R^{4a} are independently H, alkyl, alkenyl, alkynyl, or —C(═O)R⁺; and
R⁷ is alkyl, (aryl)alkyl, (heteroaryl)alkyl, (heterocyclo)alkyl, aryl, heteroaryl, or heterocylo wherein groups other than hydrogron are optionally independently substituted with one or more halo, nitro, cyano, haloalkyl, cycloheteroalkyl, —NR′R′, —OR*, —C(═O)R⁺, C(═O)OR*, C(═O)NR′R′, or —N(R′)—C(═O)(R⁺)

3. A compound of claim 2 wherein
R⁴ and R^{4a} are independently H, alkyl, alkenyl, alkynyl, or —C(═O)R⁺; and
R⁷ is phenyl or pyridyl optionally independently substituted with one to three halo, cyano, nitro, —NR′R′, —OR*, —C(═O)R⁺, C(═O)OR*, C(═O)NR′R′, or —N(R′)—C(═O)(R⁺)

4. A compound of claim 3 wherein R⁷ is phenyl independently substituted with one to three halo, cyano, nitro, —NR′R′, —OR*, —C(═O)R⁺, C(═O)OR*, C(═O)NR′R′, or —N(R′)—C(═O)(R⁺).

5. A compound of claim 3 wherein R⁷ is pyridyl independently substituted with one to three halo, cyano, nitro, —NR′R′, —OR*, —C(═O)R⁺, C(═O)OR*, C(═O)NR′R′, or —N(R′)—C(═O)(R⁺).

6. A compound of claim 5 wherein R⁷ is pyrid-2-yl independently substituted with one to three halo, cyano, nitro, —NR′R′, —OR*, —C(═O)R⁺, C(═O)OR*, C(═O)NR′R′, or —N(R′)—C(═O)(R⁺).

7. A compound of Formula II 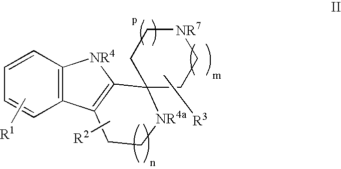
wherein
R¹, R² and R³ are each one or more optional substituents as allowed by valance at each occurrence being independently selected from
(i) halo, nitro, cyano, keto, alkyl, aryl, heteroaryl, heteroalkyl, cylcoalkyl, —NR′R′, —OR*, —C(═O)R⁺, —C(═O)OR*, —C(═O)NR′R′, —N(R′)—C(═O)(R⁺), —NR′S(O)ₓAr, —S(O)ₓAr, or —S(O)ₓNR′R′
(ii) alkyl, alkenyl, alkynyl, aryl, heteroaryl, or heteroalkyl optionally substituted with one or more groups (i) above as allowed by valence
R′ at each occurrence is independently H, alkyl, aryl, heteroaryl, hetoralkyl, cycloalkyl, (aryl)alkyl, (heteroaryl)alkyl, (cycloalkyl)alkyl, (heteroalkyl)alkyl;
R* at each a occurrence is H, alkyl, aryl, heteroaryl, hetoralkyl, cycloalkyl, (aryl)alkyl, (heteroaryl)alkyl, (cycloalkyl)alkyl, (heteroalkyl)alkyl;
R⁺ at each occurrence is H, alkyl, aryl, heteroaryl, hetoralkyl, cycloalkyl, (aryl)alkyl, (heteroaryl)alkyl, (cycloalkyl)alkyl, (heteroalkyl)alkyl;
R⁴ and R^{4a} are independently H, alkyl, alkenyl, alkynyl, or —C(═O)R⁺; and
R⁷ is alkyl, (aryl)alkyl, (heteroaryl)alkyl, (heterocyclo)alkyl, aryl, heteroaryl, or heterocylo wherein groups other than hydrogron are optionally substituted with one or more
halo, nitro, cyano, haloalkyl, cycloheteroalkyl, —NR′R′, —OR*, —C(═O)R⁺, C(═O)OR*, C(═O)NR′R′, or —N(R′)—C(═O)(R⁺)
n is 0, 1, 2 or 3;
m and p are read independently 0, 1, 2 or 3 provided that m and p can not each be 0.

8. A compound of claim 7 wherein R⁷ is phenyl or pyridyl optionally independently substituted with one to three halo, cyano, nitro, —NR′R′, —OR*, —C(═O)R⁺, C(═O)OR*, C(═O)NR′R′, or —N(R′)—C(═O)(R⁺)

9. A compound of claim 8 wherein R⁷ is phenyl independently substituted with one to three halo, cyano, nitro, —NR′R′, —OR*, —C(═O)R⁺, C(═O)OR*, C(═O)NR′R′, or —N(R′)—C(═O)(R⁺).

10. A compound of claim 8 wherein R⁷ is pyridyl independently substituted with one to three halo, cyano, nitro, —NR′R′, —OR*, —C(═O)R⁺, C(═O)OR*, C(═O)NR′R′, or —N(R′)—C(═O)(R⁺).

11. A compound of claim 10 wherein R⁷ is pyrid-2-yl independently substituted with one to three halo, cyano, nitro, —NR′R′, —OR*, —C(═O)R⁺, C(═O)OR*, C(═O)NR′R′, or —N(R′)—C(═O)(R⁺).

12. A compound of Formula III 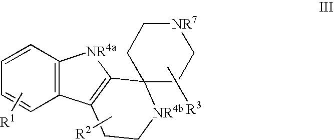
wherein
R¹, R² and R³ are each one or more optional substituents as allowed by valance at each occurrence being independently selected from
(i) halo, nitro, cyano, keto, alkyl, aryl, heteroaryl, heteroalkyl, cylcoalkyl, —NR′R′, —OR*, —C(═O)R⁺, —C(═O)OR*, —C(═O)NR′R′, —N(R′)—C(═O)(R⁺), —NR′S(O)ₓAr, —S(O)ₓAr, or —S(O)ₓNR′R′
(ii) alkyl, alkenyl, alkynyl, aryl, heteroaryl, or heteroalkyl optionally substituted with one or more groups (i) above as allowed by valence
R′ at each occurrence is independently H, alkyl, aryl, heteroaryl, hetoralkyl, cycloalkyl, (aryl)alkyl, (heteroaryl)alkyl, (cycloalkyl)alkyl, (heteroalkyl)alkyl;
R* at each a occurrence is H, alkyl, aryl, heteroaryl, hetoralkyl, cycloalkyl, (aryl)alkyl, (heteroaryl)alkyl, (cycloalkyl)alkyl, (heteroalkyl)alkyl;
R⁺ at each occurrence is H, alkyl, aryl, heteroaryl, hetoralkyl, cycloalkyl, (aryl)alkyl, (heteroaryl)alkyl, (cycloalkyl)alkyl, (heteroalkyl)alkyl;
R⁴ and R^{4a} are independently H, alkyl, alkenyl, alkynyl, or —C(═O)R⁺; and
R⁷ is alkyl, (aryl)alkyl, (heteroaryl)alkyl, (heterocyclo)alkyl, aryl, heteroaryl, or heterocylo wherein groups other than hydrogron are optionally substituted with one or more
halo, nitro, cyano, haloalkyl, cycloheteroalkyl, —NR′R′, —OR*, —C(═O)R⁺, C(═O)OR*, C(═O)NR′R′, or —N(R′)—C(═O)(R⁺)
n is 0, 1, 2 or 3;
m and p are read independently 0, 1, 2 or 3 provided that m and p can not each be 0.

13. A compound of claim 12 wherein R⁷ is phenyl or pyridyl optionally independently substituted with one to three halo, cyano, nitro, —NR′R′, —OR*, —C(═O)R⁺, C(═O)OR*, C(═O)NR′R′, or —N(R′)—C(═O)(R⁺)

14. A compound of claim 13 wherein R⁷ is phenyl independently substituted with one to three halo, cyano, nitro, —NR′R′, —OR*, —C(═O)R⁺, C(═O)OR*, C(═O)NR′R′, or —N(R′)—C(═O)(R⁺).

15. A compound of claim 13 wherein R⁷ is pyridyl independently substituted with one to three halo, cyano, nitro, —NR′R′, —OR*, —C(═O)R⁺, C(═O)OR*, C(═O)NR′R′, or —N(R′)—C(═O)(R⁺).

16. A compound of claim 15 wherein R⁷ is pyrid-2-yl independently substituted with one to three halo, cyano, nitro, —NR′R′, —OR*, —C(═O)R⁺, C(═O)OR*, C(═O)NR′R′, or —N(R′)—C(═O)(R⁺).

17. A pharmaceutical composition comprising a compound of claim 1 together with a pharmaceutically acceptable vehicle or carrier for said compound.

18. A pharmaceutical composition comprising a compound of claim 7 together with a pharmaceutically acceptable vehicle or carrier for said compound.

19. A pharmaceutical composition comprising a compound of claim 12 together with a pharmaceutically acceptable vehicle or carrier for said compound.

20. A method of treating a CXCR3-mediated disorder comprising administering to a patient in need of such treatment an effective amount of a compound of claim 1.
